(19) United States
(12) Patent Application Publication
(10) Pub. No.: US 2026/0097081 A1
(43) Pub. Date: Apr. 9, 2026

(54) PHARMACEUTICAL COMPOSITION

(71) Applicants: FUKUOKA UNIVERSITY, Fukuoka (JP); OTSUKA PHARMACEUTICAL FACTORY, INC., Tokushima (JP)

(72) Inventors: Shohta KODAMA, Fukuoka (JP); Masuhiro Nishimura, Tokushima (JP); Shinichi Matsumoto, Tokushima (JP); Osamu Sawamoto, Tokushima (JP)

(73) Assignees: FUKUOKA UNIVERSITY, Fukuoka (JP); OTSUKA PHARMACEUTICAL FACTORY, INC., Tokushima (JP)

(21) Appl. No.: 19/355,935

(22) Filed: Oct. 10, 2025

Related U.S. Application Data
(63) Continuation of application No. 17/605,412.

(30) Foreign Application Priority Data
Apr. 24, 2019 (JP) 2019-082768

(51) Int. Cl. A61K 35/28; A61P 9/10; A61P 17/02

(52) U.S. Cl. CPC A61K 35/28; A61P 9/10; A61P 17/02

(57) ABSTRACT

An object of the present invention is to provide a pharmaceutical composition containing mesenchymal stem cells that exhibit excellent therapeutic effects on various diseases, injured parts, wounds and decubitus. The present invention relates to a pharmaceutical composition for treating a non-porcine animal, the pharmaceutical composition includes a neonatal pig-derived mesenchymal stem cell which produces at least one humoral factor selected from TGF-β1, TGF-β2, VEGF-A and VEGF-C.

### CROSS-REFERENCE TO RELATED APPLICATIONS

This application is a Continuation of U.S. application Ser. No. 17/605,412, filed on Oct. 21, 2021, which is a National Phase Entry of PCT International Application No. PCT/JP2020/005525, filed on Feb. 13, 2020, which claims priority to Japanese Patent Application No. 2019-082768, filed on Apr. 24, 2019, the contents of all of which are incorporated herein by reference in their entirety.

### TECHNICAL FIELD

The present invention relates to a pharmaceutical composition for treating non-porcine animals, and more particularly, to a pharmaceutical composition for treating a non-porcine animal containing mesenchymal stem cells derived from a neonatal pig.

### BACKGROUND ART

Due to recent advances of research in somatic stem cells including mesenchymal stem cells, clinical application of somatic stem cells has already been shifted from the basic research stage to the development stage. Somatic stem cells have three major functions (pluripotency, immunoregulatory ability, and remodeling ability in the extracellular environment), and are expected as cells for treating a refractory disease.

Firstly, the pluripotency is an ability of somatic stem cells to directly differentiate into bone, cartilage, or the like, and administered somatic stem cells complement lost cells or substitute for cells having an insufficient function, thereby exhibiting a therapeutic effect.

Secondly, the immunoregulatory ability works on immunocompetent cells of a patient through secretion of an anti-inflammatory cytokine, chemokine, exosome, or the like from somatic stem cells or through an intercellular adhesion molecule or the like so as to suppress an inflammation or an immunoreaction in a graft-versus-host disease or the like, thereby exhibiting a therapeutic effect.

Thirdly, with respect to the remodeling ability in the extracellular environment, a therapeutic effect is exhibited by secretion of an angiogenesis promoting factor, a vascular inducing factor, a growth factor, an antifibrotic factor, or the like from somatic stem cells in an infarct site in an ischemic disease, a fibrotic site caused by an inflammation, or the like.

Mesenchymal stem cells are somatic stem cells present in the bone marrow, fat, pancreatic islet, umbilical cord blood, and the like of a mammal, and derived from a mesodermal tissue (mesenchyme) and have an ability to differentiate into cells belonging to the mesenchymal lineage. Recently, a clinical trial has been carried out for diseases such as a graft-versus-host disease, a cardiovascular disorder, an autoimmune disease, osteoarthritis, dysostosis, a hepatic disorder, a respiratory disease, spinal cord injury, cerebral infarction, and renal failure (Non-Patent Literature 1), and mesenchymal stem cells have been expected to be used in various clinical applications (Non-Patent Literature 2). However, the effects of these clinical applications are not sufficient.

In addition, in clinical applications of mesenchymal stem cells, there are problems such as with respect to securing of a donor, invasion in a donor, and security of safety such as a virus free test for each donor. Since the effect of mesenchymal stem cells varies largely depending on the donor or the conditions such as the age of the donor, securing of stable quality is also a big problem (Non-Patent Literature 3).

Angiogenesis is controlled in a complicated manner under the balance of a variety of an angiogenesis promoting factor, an inhibiting factor, metalloprotease, or other enzymes, and is deeply related to wound healing or various diseases. In angiogenesis, there are physiological phenomena observed by, for example, formation of osteoblasts at the time of wound, and pathological phenomena such as inflammatory diseases and angiogenesis diseases such as arteriosclerosis (Non-Patent Literature 4).

Lymphatic vessels form a wide area network together with the blood vessels in the living body, absorbs the interstitial fluid, protein, fat or immunocharge cell, or the like, which has leaked from the blood vessel in the peripheral tissue, and maintain the closed circulation system of the blood vessels by circulating to a vascular system via the collecting lymphatic vessel. The induction of lymphangiogenesis and the induction of angiogenesis has been observed in the healing wounds and various pathological inflammations (Non-Patent Literature 5).

### RELATED ART

### Non-Patent Literature

Non-Patent Literature 1: Lemos N E, de Almeida Brondani L, Dieter C, Rheinheimer J, Boucas A P, Bauermann Leitao C, Crispim D, Bauer A C. Islets. 2017 Jul. 5: e1335842. doi: 10.1080/19382014.2017.1335842
Non-Patent Literature 2: Tomohiko Kazama, Journals of Nihon University Medical Association, Vol. 75, No. 2, Pages 61 to 66, 2016
Non-Patent Literature 3: Hajime Ohgushi, Biochemistry, Vol. 81, No. 2, Pages 99 to 104, February 2009
Non-Patent Literature 4: Mayumi Ono et al., Chemistry and Biology, Vol. 37, No. 1, Pages 14 to 19, 1999
Non-Patent Literature 5: Kanako Hosono et al., Folia Pharmacologica Japonic, No. 141, Pages 290 to 291, 2013

### SUMMARY OF INVENTION

### Problems to be Solved by the Invention

In view of the above circumstances, an object of the present invention is to provide a pharmaceutical composition containing mesenchymal stem cells that exhibit excellent therapeutic effects on various diseases, injured parts, wounds and decubitus.

### Problems to be Solved by the Invention

### Means for Solving the Problems

The present inventors have found that mesenchymal stem cells prepared from a neonatal pig highly express a specific humoral factor, and have a small cell size and excellent proliferation ability as compared with conventional mesenchymal stem cells, and thus completed the present invention.

That is, the present invention relates to the following.
1. A pharmaceutical composition for treating a non-porcine animal, the pharmaceutical composition comprising:
   a neonatal pig-derived mesenchymal stem cell which produces at least one humoral factor selected from a transformation growth factor-β (hereinafter, referred to as “TGF-β”) 1, TGF-β2, and a vascular endothelial growth factor (hereinafter, referred to as “VEGF”)-A and VEGF-C.
2. The pharmaceutical composition according to above 1, wherein the non-porcine animal is treated by the promotion of angiogenesis and/or lymphangiogenesis.
3. The pharmaceutical composition according to above 1 or 2, wherein at least one selected from peripheral artery diseases, cerebral infarction, myocardial infarction, acute lung injury, wounds, skin injury, and decubitus is treated.
4. The pharmaceutical composition according to any one of above 1 to 3, wherein the neonatal pig-derived mesenchymal stem cell is derived from a fetal pig or a pig less than one month after birth.
5. The pharmaceutical composition according to any one of above 1 to 4, wherein the neonatal pig-derived mesenchymal stem cell is derived from a fetal pig or a pig less than 25 days after birth.
6. The pharmaceutical composition according to any one of above 1 to 5, wherein the non-porcine animal is a human.

### Effects of the Invention

Since the pharmaceutical composition of the present invention contains neonatal pig-derived mesenchymal stem cells, the action of the humoral factor produced by the neonatal pig-derived mesenchymal stem cells produces an excellent treatment effect on various diseases, injured parts, wounds and decubitus.

### BRIEF DESCRIPTION OF DRAWINGS

FIG. 1A is a view showing a total cell amount for a specific culture period (days) when stem cells of the present invention are cultured. FIG. 1B is a view showing a total cell growth rate for a specific culture period (days) when the stem cells of the present invention are cultured. In FIG. 1A and FIG. 1B, a dotted line and black circles show neonatal pig bone marrow-derived mesenchymal stem cells (hereinafter, also abbreviated as npBM-MSC), and a solid line and white circles show human bone marrow-derived mesenchymal stem cells (hereinafter, also abbreviated as hBM-MSC).

FIG. 2A shows results of measuring the concentration of TGF-β1 in a culture supernatant of npBM-MSC and mouse bone marrow-derived mesenchymal stem cells (hereinafter, also abbreviated as mBM-MSC). FIG. 1B shows results of measuring the concentration of TGF-β2 in the culture supernatant of npBM-MSC and mBM-MSC.

FIG. 3A shows results of measuring the concentration of VEGF-A in the culture supernatant of each of npBM-MSC and mBM-MSC. FIG. 3B shows results of measuring the concentration of VEGF-C in the culture supernatant of npBM-MSC and mBM-MSC.

FIG. 4 shows results of the evaluation of the blood flow by intramuscular injection of npBM-MSC into the thigh muscle tissue of the ischemic limb.

FIG. 5 shows results of the evaluation of the blood flow by intramuscular injection of npBM-MSC or mBM-MSC into the thigh muscle tissue of the ischemic limb.

### DESCRIPTION OF EMBODIMENTS

The mesenchymal stem cell is a somatic stem cell derived from a mesodermal tissue (mesenchyme), and refers to a cell that has an ability to differentiate into cells belonging to mesenchymal systems such as osteocytes, cardiomyocytes, chondrocytes, tendon cells, and adipocytes, and can proliferate while maintaining the differentiation ability. The neonatal pig-derived mesenchymal stem cells in the present invention may be mesenchymal stem cells isolated from a neonatal pig, and include, for example, mesenchymal stem cells derived from the bone marrow; the pancreatic islet, the skin, fat, or the like of a neonatal pig.

In the present invention, the “neonatal pig” refers to a fetal pig or a pig less than one month after birth, preferably less than 25 days after birth. The neonatal pig is preferably a pig for medical use, and more preferably a neonatal pig that enables cell transplantation into a human. The breed of the pig is not particularly limited, however, examples thereof include Landrace breed (for example, Danish Landrace breed, American Landrace breed, British Landrace breed, Dutch Landrace breed, and Swedish Landrace breed), Large White Yorkshire breed, Berkshire breed, Duroc breed, Hampshire breed, Middle White Yorkshire breed, and a miniature pig, and above all, Landrace breed is preferred.

As long as the neonatal pig-derived mesenchymal stem cells in the present invention are mesenchymal stem cells isolated from a neonatal pig, stem cells that are primary cultured cells thereof and cells obtained by subculturing the primary cultured cells, and can generate various types of cells expressing various types of differentiation markers are also included in the mesenchymal stem cells of the present invention.

The neonatal pig-derived mesenchymal stem cells in the present invention produce at least one humoral factor selected from TGF-β1, TGF-β2, VEGF-A and VEGF-C, and preferably produce at least TGF-β1, TGF-β2, and VEGF-C thereof.

The TGF-β is a cytokine family having a second half biological activity, and therefore, there are three isoforms TGF-β1, 2, and 3 having high structural homology in mammals. The TGF-β has an action of promoting angiogenesis and an action of promoting lymphangiogenesis.

The VEGF is a cytokine family that specifically acts on vascular endothelial cells, and there are seven types of, respectively; VEGF-A, VEGF-B, VEGF-C, VEGF-D, VEGF-E, PlGF (placental growth factor)-1, and PlGF-2. The VEGF has an action of promoting angiogenesis and an action of promoting lymphangiogenesis.

The pharmaceutical composition of the present invention contains neonatal pig-derived mesenchymal stem cells which produce at least one humoral factor selected from TGF-β1, TGF-β2, VEGF-A, and VEGF-C, whereby at least one of angiogenesis and lymphangiogenesis can be promoted by the action of the humoral factor produced from the cells, and the non-porcine animal can be treated.

Examples of the treatment by promoting at least one of angiogenesis and lymphangiogenesis include at least one treatment selected from a disease treatment, an injury treatment, and wound healing, and preferably at least one treatment selected from peripheral artery diseases, cerebral infarction, myocardial infarction, acute lung injury, wounds, skin injury, and decubitus.

The non-porcine animal is not particularly limited as long as the animal is an animal other than a pig, and is preferably a mammal other than a pig, and examples thereof include humans, mice, rats, hamsters, guinea pigs, rabbits, dogs, cats, horses, cows, sheep, goats, a marmoset, a monkey, and the like.

The production of the humoral factor of the neonatal pig-derived mesenchymal stem cells in the present invention preferably exhibit high expression. Here, “high expression” means that the expression level of a humoral factor is equal to or more than that of conventional mesenchymal stem cells. Here, examples of the conventional mesenchymal stem cells include mouse bone marrow-derived mesenchymal stem cells, which will be described later in Examples.

In the neonatal pig-derived mesenchymal stem cells in the present invention, the expression level of the humoral factor is significantly higher than that of the mouse bone marrow-derived mesenchymal stem cells, and the expression intensity of the protein is preferably 1.1 or more, more preferably 1.2 or more, and still more preferably 1.3 or more, as compared with the mouse bone marrow-derived mesenchymal stem cells. The expression intensity of the protein can be confirmed by, for example, FACS analysis using a specific antibody, ELISA, or the like.

Preferred combinations of the humoral factors that the neonatal pig-derived mesenchymal stem cells in the present invention exhibit high expression include, for example, TGF-β1, TGF-β2, and VEGF-C. Specifically, for example, the expression levels of TGF-β1 and TGF-β2 after culturing the neonatal pig-derived mesenchymal stem cells in the present invention for 3 days in the MSC medium described later are preferably 1.1 times or more, more preferably 1.5 times or more, and still more preferably 2 times or more as compared with the mouse bone marrow-derived mesenchymal stem cells which are cultured under the same conditions. In addition, for example, the expression level of VEGF-C after culturing the neonatal pig-derived mesenchymal stem cells in the present invention for 3 days in the below-mentioned MSC minimal essential medium is preferably 1.1 times or more, more preferably 1.2 times or more, and still more preferably 1.3 times or more as compared with the mouse bone marrow-derived mesenchymal stem cells which are cultured under the same conditions.

In the neonatal pig-derived mesenchymal stem cells in the present invention, it is preferred that 60% or more of the cells are positive for CD44 and CD90 that are cell markers, more preferably 70% or more, and further more preferably 80% or more of the cells are positive for CD44 and CD90. Further, it is preferred that 60% or more of the cells are positive for CD29 that is a cell marker, more preferably 70% or more, and further more preferably 80% or more of the cells are positive for CD29. The positive rate of the cell marker can be confirmed by a method using flow cytometry or the like as will be described later in Examples.

In the neonatal pig-derived mesenchymal stem cells in the present invention, a doubling time in a logarithmic growth phase is preferably 36 hours or less, more preferably 32 hours or less, further more preferably 28 hours or less, particularly preferably 24 hours or less, and most preferably 20 hours or less. In addition, the doubling time in a logarithmic growth phase is preferably 14 hours or more, and more preferably 16 hours or more.

The culture in a logarithmic growth phase of the neonatal pig-derived mesenchymal stem cells in the present invention can be performed by inoculating the stem cells of the present invention into a medium containing vitamin C described below (for example, MSC medium) and culturing the cells in an incubator for culture at 37° C. in the presence of 5% CO₂. As the doubling time in the logarithmic growth phase is shorter, it becomes possible to prepare a large amount of neonatal pig-derived mesenchymal stem cells in a short time and at low cost.

The neonatal pig-derived mesenchymal stem cells in the present invention have an average diameter of preferably 17 μm or less, more preferably 16.5 μm or less, further more preferably 16 μm or less, particularly preferably 15.5 μm or less, and most preferably 15 μm or less. The average diameter is preferably 10 μm or more, more preferably 12 μm or more. As the average diameter is smaller, formation of pulmonary embolism by administration of the neonatal pig-derived mesenchymal stem cells can be prevented. The average diameter can be measured using, for example, Nucleo Counter NC-200 (trademark). Here, the average refers to arithmetic average.

In the differentiation from the neonatal pig-derived mesenchymal stem cells in the present invention into adipocytes, for example, by culturing the neonatal pig-derived mesenchymal stem cells in the present invention in the presence of insulin, MCGS (a serum component, Mesenchymal Stem Cell Growth Supplement), dexamethasone, indomethacin, isobutyl methylxanthine, and the like, the differentiation into adipocytes can be induced.

In the differentiation into adipocytes and the maintenance, a commercially available kit or medium, or the like may be used, and examples thereof include hMSC differentiation BulletKit (trademark)-adipogeni (PT-3004) manufactured by Lonza Walkersville, Inc., hMSC adipogenic induction medium (PT-3102B) manufactured by Lonza Walkersville, Inc., hMSC adipogenic maintenance medium (PT-3102B) manufactured by Lonza Walkersville, Inc., and the like. The differentiation from neonatal pig-derived mesenchymal stem cells into adipocytes can be confirmed using a commercially available kit, and examples thereof include Adipo Red (trademark) assay reagent manufactured by Lonza, Inc.

In the differentiation from the neonatal pig-derived mesenchymal stem cells in the present invention into osteocytes, for example, by culturing the neonatal pig-derived mesenchymal stem cells in the present invention in the presence of dexamethasone, an ascorbate salt, MCGS, β-glycerophosphoric acid, and the like, the differentiation into osteocytes can be induced. In addition, a commercially available kit may be used, and examples thereof include hMSC differentiation BulletKit (trademark)-osteogenic, PT-3004 manufactured by Lonza Walkersville, Inc., and the like. The differentiation from neonatal pig-derived mesenchymal stem cells into osteocytes can be confirmed using a commercially available alkaline phosphatase staining kit (for example, manufactured by Cosmo Bio Co., Ltd., or the like), a commercially available calcification staining kit (for example, manufactured by Cosmo Bio Co., Ltd., or the like), or the like.

In the differentiation from the neonatal pig-derived mesenchymal stem cells in the present invention into chondrocytes, for example, by culturing the neonatal pig-derived mesenchymal stem cells in the present invention in the presence of TGF-β3, dexamethasone, insulin-transferrin-selenious acid (ITS), sodium pyruvate, proline, and an ascorbate salt, the differentiation into chondrocytes can be induced. In addition, a commercially available kit may be used, and examples thereof include hMSC differentiation BulletKit (trademark)-condrogenic, PT-3003 manufactured by Lonza Walkersville, Inc., and the like. The differentiation from neonatal pig-derived mesenchymal stem cells into chondrocytes can be confirmed by Alcian blue staining, or the like.

The method for producing a pharmaceutical composition according to the present invention includes a step of preparing neonatal pig-derived mesenchymal stem cells. One embodiment of the method for preparing neonatal pig-derived mesenchymal stem cells in the present invention is, for example, the method comprising the following steps.
(1) a step of collecting cells from a neonatal pig
(2) a step of preparing neonatal pig-derived mesenchymal stem cells by culturing the cells collected in the step (1)

Hereinafter, the respective steps will be described.

(1) Step of Collecting Cells from Neonatal Pig

In the step (1), cells are collected from the bone marrow; fat, skin, pancreas, or the like of a neonatal pig. Specifically, for example, when cells are collected from the bone marrow of a neonatal pig, bone marrow cells can be collected from the femur, iliac crest, breast bone, or the like of a neonatal pig. For example, the femur is collected from a neonatal pig, both ends are cut off, a needle is inserted, washing off is performed with a physiological buffer solution (for example, a phosphate buffer solution, hereinafter also referred to as PBS) supplemented with heparin, and an outflow liquid from a place on the opposite side is recovered as a bone marrow liquid. When the amount of the outflow liquid is decreased, the bone is reversed and the needle is inserted on the opposite side, and then washing off is performed again with PBS and a bone marrow liquid that is a cell-containing solution is prepared.

Further, a neonatal pig-derived monocytic cell fraction may be isolated by conventional centrifugation of the cell-containing solution prepared above. The cell-containing solution prepared above is diluted with PBS or the like, and in a tube containing a medium for separation of human lymphocytes (for example, Ficoll-Paque PLUS manufactured by GE Healthcare Life Sciences, or the like), the diluted cell-containing solution is placed on a layer of the medium.

The tube is centrifuged to separate layers, and a layer containing neonatal pig-derived monocytic cells is recovered. The recovered solution is further centrifuged, and the supernatant is removed, then, the resultant is diluted with PBS or the like, followed by centrifugation again, whereby a monocytic cell fraction is isolated. The cells in the monocytic cell fraction isolated in this manner may be cryopreserved before culture. By freezing the cells in the isolated neonatal pig-derived monocytic cell fraction, cells that are less likely to be affected by freezing and thawing can be selectively prepared. When the cells are cryopreserved before culture, the temperature is preferably −80° C. or lower, and more preferably −150° C. or lower.

Further, for example, when cells are collected from the pancreas of a neonatal pig, the pancreatic islet is collected from a neonatal pig, furthermore, in some cases, the pancreatic islet is subjected to suspension culture, whereby a cell cluster to be used in adhesion culture for the purpose of preparing stem cells is prepared.

Further, for example, when cells are collected from the fat of a neonatal pig, the fat is collected from a neonatal pig and cut into fine pieces with scissors, followed by an enzyme treatment. The resultant is filtered through a cell strainer, and then centrifuged at a low speed. The cells precipitated on the bottom of the tube are used for culture. In addition, for example, when cells are collected from the skin (including hair) of a neonatal pig, the skin is collected from a neonatal pig and is subjected to an enzyme treatment. After the enzyme treatment, the hair is removed from the skin, and a bulge portion is collected and used for culture. When culture is performed, 3T3 feeder cells are used.

### (2) A Step of Preparing Neonatal Pig-Derived Mesenchymal Stem Cells by Culturing the Cells Collected in the Step (1)

In the cells, the cell fraction, or the cell cluster collected in the above step (1), unintended cells other than stem cells are contained in a large amount. In general, a culture method in which such cells are removed by using a minimal essential medium not containing vitamin C, which is essential for survival of such unintended cells (for example, the below-mentioned MSC minimal essential medium) is used.

In the step (2) of the present invention, the cells, the cell fraction, or the cell cluster collected in the above step (1) are/is cultured in an incubator for culture preferably at 35° C. to 39° C., more preferably at 36° C. to 38° C., and most preferably at 37° C. in the presence of CO₂ preferably at 4 to 6%, more preferably at 4.5 to 5.5%, and most preferably at 5%, whereby the unintended cells which are other than mesenchymal stem cells are removed, and also the neonatal pig-derived mesenchymal stem cells in the present invention are proliferated.

The neonatal pig-derived mesenchymal stem cells in the present invention have a significantly high growth rate, and therefore, the neonatal pig-derived mesenchymal stem cells in the present invention can be prepared by using only a medium containing vitamin C (for example, the below-mentioned MSC medium), instead of using a minimal essential medium not containing vitamin C, for culture to remove the above-mentioned unintended cells. Incidentally, the neonatal pig-derived mesenchymal stem cells in the present invention can also be prepared by culture using a minimal essential medium not containing vitamin C for removing the above-mentioned unintended cells, and thereafter replacing the medium with a medium containing vitamin C so as to proliferate the neonatal pig-derived mesenchymal stem cells in the present invention.

The neonatal pig-derived mesenchymal stem cells in the present invention are cultured by; specifically, for example, the following method. A container for culture coated with gelatin (for example, a plate coated with 0.1% gelatin) or a container for culture without gelatin coat (for example, a plate) is used, and a minimal essential medium not containing vitamin C (for example, the below-mentioned MSC minimal essential medium) or a medium containing vitamin C (for example, the below-mentioned MSC medium) is used, and primary cultured cells are obtained by inoculating the cells preferably at 5.0×10⁵ cells to 5.0×10⁷ cells/9.6 cm², and incubating the cells, for example, under the conditions of 37° C., 5% CO₂, and 90% humidity:

The culture period for obtaining the primary cultured cells is preferably 3 to 12 days, more preferably 3 to 11 days, and most preferably 3 to 10 days after inoculation. The primary cultured cells may be subcultured. The stem cells obtained by subculture are also referred to as subcultured cells.

The subculture of the primary cultured cells or the subcultured cells is performed after the stem cells reached 30% to 100% confluence, preferably 50% to 95% confluence, more preferably 60% to 90% confluence, and most preferably 70% to 85% confluence preferably after 2 days to 6 days, more preferably after 2 days to 5 days, further more preferably after 2 days to 4 days, and most preferably after 3 days from the inoculation of the stem cells.

In the inoculation of the stem cells, the cells are inoculated preferably at 5.0×10⁵ cells/9.6 cm² to 5.0×10⁷ cells/9.6 cm² using a container for culture coated with gelatin (for example, a plate coated with 0.1% gelatin) or a container for culture without gelatin coat (for example, a plate), and using a medium containing vitamin C (for example, the below-mentioned MSC medium). In the culture of the stem cells, the cells are cultured, for example, under the conditions of 37° C., 5% CO₂, and 90% humidity. The neonatal pig-derived mesenchymal stem cells in the present invention are proliferated by replacing a medium as needed during the culture of the neonatal pig-derived mesenchymal stem cells.

As the MSC minimal essential medium and the MSC medium, conventionally known media can be used, and commercially available media may be used. Examples of the MSC minimal essential medium include a medium obtained by adding 55 mL of fetal bovine serum (FBS) manufactured by Gibco, Inc. and 5.5 mL of penicillin-streptomycin manufactured by Sigma-Aldorich Co. LLC to 500 mL of MEMα (nucleosides, no ascorbic acid) manufactured by Gibco, Inc. Further, examples of the MSC medium include a medium obtained by adding 55 mL of fetal bovine serum (FBS) manufactured by Gibco, Inc., 5.5 mL of penicillin-streptomycin manufactured by Sigma-Aldorich Co. LLC, and 22.2 μL of FGF-Basic, recombinant, expressed in E. coli, suitable for cell culture (final concentration: 1 ng/ml) manufactured by Sigma-Aldorich Co. LLC to 500 mL of MEMα (nucleosides) manufactured by Gibco, Inc.

The subculture is preferably performed at least one or more times. The number of subcultures is not particularly limited as long as the neonatal pig-derived mesenchymal stem cells in the present invention are obtained, but is preferably 1 to 3, more preferably 1 to 20.

The neonatal pig-derived mesenchymal stem cells in the present invention can be cryopreserved. The timing of cryopreservation is not particularly limited, but is preferably after 1 subculture to 20 subcultures, more preferably after 2 subcultures to 10 subcultures. As the cryopreserving and thawing methods, conventionally known methods can be used.

As the method for cryopreserving the neonatal pig-derived mesenchymal stem cells in the present invention, specifically; for example, the cells are dispersed in a cryopreservation solution, and can be cryopreserved at −80° C. or lower in a freezer or in liquid nitrogen until they are needed. Examples of the cryopreservation solution include a solution obtained by mixing OPF-301 [a Ringer's lactate solution containing 3% trehalose and 5% dextran (WO 2014/208053)] and dimethyl sulfoxide (DMSO) at a ratio of 9:1, a serum-containing or serum-free preservation solution that can be used for cryopreservation of animal cells, or a commercially available reagent for cell cryopreservation [preferably, a cell banker such as CELLBANKER (registered trademark) manufactured by Takara Bio, Inc.].

The pharmaceutical composition of the present invention may contain other components than the neonatal pig-derived mesenchymal stem cells on the condition that the expected treatment effect is maintained. Examples of the components that can be used in the pharmaceutical composition of the present invention include an organic bioabsorbable material such as hyaluronic acid, collagen and fibrinogen, a gelling material such as hyaluronic acid, collagen (for example, soluble collagen such as acid-soluble collagen, alkali-soluble collagen, and enzyme-soluble collagen) and fibrin glue, and an aqueous solvent such as a buffer solution such as sterile water, physiological saline, or phosphate solution. In addition to these components, antibiotics, stabilizers, preservatives, pH adjusters, humoral factors, and the like may be included.

The administration method when the pharmaceutical composition of the present invention is used as a pharmaceutical product is not particularly limited, but intramuscular administration, subcutaneous administration, intravascular administration (preferably intravenous administration), intraperitoneal administration, intraintestinal administration, and the like are preferred, and intramuscular administration, subcutaneous administration, and intravascular administration are more preferred.

The dosage of the pharmaceutical composition of the present invention may vary depending on the state of the patient (for example, body weight, age, symptoms, physical condition, and the like), from the viewpoint of achieving a sufficient preventive or therapeutic effect, it is preferred that the amount is large, and on the other hand, from the viewpoint of suppressing side effects, it is preferred that the amount is small.

Usually, in the case of administration to an adult, the number of neonatal pig-derived mesenchymal stem cells is from 5×10²/times to 1×10¹²/times, preferably from 1×10⁴/times to 1×10¹¹/times, and more preferably from 1×10⁵/times to 1×10¹⁰/times. Incidentally, this dose may be administered as a single dose in a plurality of times, or this dose may be administered in a plurality of divided doses.

Usually, in the case of administration to an adult, the number of neonatal pig-derived mesenchymal stem cells per body weight is 1×10/kg to 5×10¹⁰/kg, preferably 1×10²/kg to 5×10⁹/kg, and more preferably 1×10³/kg to 5×10⁸/kg. Incidentally, this dose may be administered as a single dose in a plurality of times, or this dose may be administered in a plurality of divided doses.

### EXAMPLES

### Reference Example 1

### Recovery of Neonatal Pig-Derived Bone Marrow Cells

The bone marrow was collected from the femur of a neonatal pig. The femur was collected from a neonatal pig (a medical Landrace breed pig, 23 days after birth), both ends were cut off, a 12 G needle was inserted, washing off was performed with 50 mL of PBS treated with heparin (3 mL of heparin (1000 U/mL) and 47 mL of PBS), and 50 mL of an outflow liquid of the bone marrow (hereinafter also abbreviated as bone marrow liquid) was recovered from a place on the opposite side. When the amount of the outflow liquid was decreased, the bone was reversed and the needle was inserted on the opposite side, and then washing off was performed again with PBS and a bone marrow liquid was collected. In a 15-mL conical tube for counting, 50 μL of a sample was taken in 1950 μL of PBS (40-fold dilution), and the number of cells was measured using a cell counter.

[Isolation of Neonatal Pig-Derived Monocytic Cell (npMNC) Fraction]

The bone marrow liquid obtained by the above-mentioned procedure was calmly resuspended. The entire bone marrow liquid was dispensed into four 50-mL tubes in an amount of 10 mL each, and each liquid was diluted to 30 mL with PBS and mixed well while confirming that the cells did not adhere to the tube. To four new 50-mL tubes, 10 mL of Ficoll-Paque PLUS (manufactured by GE Healthcare Life Sciences) was added, and 30 mL of the bone marrow liquid mixed with PBS was placed on the layer of Ficoll-Paque PLUS.

Each of the tubes was centrifuged at 20° C. for 30 minutes at 400×g, the speed was slowly accelerated (⅓ the full speed) without applying the break, whereby three different layers were formed. The monocytic cell fraction was positioned in a suspended white ring, and therefore, the entire white ring was collected in a 50-mL tube (×4) containing 25 mL of PBS. Centrifugation was performed at room temperature for 7 minutes at 400×g, and the supernatant was removed. PBS was added up to 40 mL, and centrifugation was performed again at room temperature for 7 minutes at 400×g. When the number of cells was measured in the same manner as described above, 25% to 30% cells were isolated as the monocytic cell fraction ((20 to 30)×10⁶ cells each) among the entire bone marrow cells.

[Cryopreservation of Cells in npMNC Fraction]

The isolated cells in the monocytic cell fraction were placed in a cryovial containing FBS mixed with DMSO (90% FBS and 10% DMSO) at 10⁷ cells/mL, and the total volume of the cell suspension was set to 1 mL [cell number/10×10⁶=the volume (mL) of FBS mixed with DMSO]. The cryovial was stored at −20° C. for 1 hour, and subsequently stored at −80° C. for 24 hours, and then finally transferred to a liquid nitrogen tank for long-term storage.

[Culture of Cells in npMNC Fraction and Preparation of npBM-MSC]

The cell suspension containing the cells in the npMNC fraction cryopreserved in the cryovial was promptly thawed in a water bath at 37° C., and the thawed cell suspension was calmly added to 30 mL of MSC minimal essential medium [a medium obtained by adding 55 mL of fetal bovine serum (FBS) manufactured by Gibco, Inc. and 5.5 mL of penicillin-streptomycin manufactured by Sigma-Aldorich Co. LLC to 500 mL of MEMα (nucleosides, no ascorbic acid) manufactured by Gibco, Inc., hereinafter the same shall apply] adjusted to reach the temperature equilibrium (37° C.) using a micropipette. The resultant was centrifuged at room temperature for 5 minutes at 500×g, and the resulting pellet was resuspended in 4 mL of the temperature-equilibrated MSC minimal essential medium, and the suspension was gently pipetted up and down. As a result of measurement of the total number of cells and the number of viable cells, the total number of cells was 4.18×10⁶, the number of viable cells was 6.6×10⁵, and the viability was 15.8%.

A 6-well plate was coated with 0.1% gelatin and left to stand for 10 to 15 minutes in an incubator (37° C., 5% CO₂), and thereafter, the gelatin was removed before use. The cell suspension was added to each of the prepared 0.1% gelatin-coated 6-well plate, and the cell suspension was dispersed on a growth surface (gelatin coat) by gently shaking, and the cells were inoculated into 2 mL of the MSC minimal essential medium at 2.09×10⁶ cells/well. In a CO₂ incubator, the cells were cultured under the conditions of 37° C., 5% CO₂, and 90% humidity, and after 3 days, the medium was replaced with MSC medium [a medium obtained by adding 55 mL of fetal bovine serum (FBS) manufactured by Gibco, Inc., 5.5 mL of penicillin-streptomycin manufactured by Sigma-Aldorich Co. LLC, and 22.2 μL of FGF-Basic, recombinant, expressed in E. coli, suitable for cell culture (final concentration: 1 ng/ml) manufactured by Sigma-Aldorich Co. LLC to 500 mL of MEMα (nucleosides) manufactured by Gibco, Inc., hereinafter the same shall apply] so as to proliferate the cells, thereafter, the MSC medium was replaced with a fresh one every three days. The npBM-MSC reached confluence after 10 days. Also in the case of using a plate without gelatin coat, the npBM-MSC reached confluence after 10 days in the same manner.

### [Subculture]

After the npBM-MSC reached nearly 100% confluence, the cells were recovered from two wells and reinoculated into a T75 flask with or without 0.1% gelatin coat.

The cells were washed with 2 mL of PBS (not containing calcium and magnesium), 320 μL of 0.25% trypsin was added per well, and the cells were left to stand for a few minutes in an incubator, after the cells were peeled off, the medium was neutralized with 1680 μL of the MSC medium. The cell suspension was collected in a 50-mL tube using a 1-mL pipette, and 16 mL (8 mL×2 wells) of the MSC medium was added thereto, followed by centrifugation at room temperature for 5 minutes at 500×g. The obtained pellet was gently resuspended in the temperature-equilibrated MSC medium (2 mL) using a pipette As a result of measurement of the total number of cells and the number of viable cells, the total number of cells was 2.05×10⁶, the number of viable cells was 2.02×10⁶, and the viability was 98.5%.

The MSC medium was added to T75 flasks with and without 0.1% gelatin coat, and the cells were reinoculated into the T75 flasks at 4.5×10⁵ viable cells/flask, and in a CO₂ incubator, the cells were cultured under the conditions of 37° C., 5% CO₂, and 90% humidity. The cells were defined as a first subculture. After 3 days from the inoculation of the first subculture, 100% confluence was reached regardless of the presence or absence of the 0.1% gelatin coat.

[Preparation of npBM-MSC]

After the npBM-MSC reached nearly 100% confluence, the cells were recovered from two flasks of the T75 flasks with or without 0.1% gelatin coat. The cells were washed with 8 mL of PBS (−), 2.4 mL of 0.25% trypsin was added per well, and the cells were left to stand for a few minutes in an incubator, after the cells were peeled off, the medium was neutralized with 12.6 mL of the MSC medium. The cell suspension was collected in a 50-mL tube, followed by centrifugation at room temperature for 5 minutes at 500×g.

To the obtained pellet, the temperature-equilibrated MSC medium (10 mL) was added, and the pellet was calmly resuspended by pipetting up and down, and the results of measurement of the total number of cells and the number of viable cells are shown below.

The cells from the flask (×2) coated with 0.1% gelatin: the total number of cells: 1.62×10⁷, the number of viable cells: 1.60×10⁷, and the viability: 98.8%

The cells from the flask (×2) without gelatin coat: the total number of cells: 1.48×10⁷, the number of viable cells: 1.46×10⁷, and the viability: 98.6%

[Cryopreservation of npBM-MSC]

Apart from the above-mentioned culture, the early subculture npBM-MSC were frozen, whereby a cell stock was prepared. An npBM-MSC pellet that was treated with trypsin in a solution obtained by mixing CELLBANKER (registered trademark) 1 or OPF-301 [a Ringer's lactate solution containing 3% trehalose and 5% dextran (WO 2014/208053)] at a desired concentration and DMSO at a ratio of 9:1 was resuspended to 1.5×10⁶ cells/mL/vial. The vial was placed in a Vi-CELL and stored at −80° C. for 24 hours, and thereafter, the cells were transferred to liquid nitrogen from −80° C. and stored for a long period of time.

### [CFU Assay]

Into a 21-cm² culture dish (without gelatin coat or with 0.1% gelatin coat), 630 cells of the npBM-MSC (P2) were inoculated at a density of 30 cells/cm², and the cells were cultured in the MSC medium. The MSC medium was replaced with a fresh one every three days. After culture for 6 days, adherent cells were washed twice with 4 mL of PBS, and then fixed with 4 mL of ice-cooled methanol at 4° C. for 15 minutes. In order to visualize colonies, the cells were stained for 30 minutes with 4 mL of Giemsa diluted to 1:19 with a phosphate buffer solution, and thereafter washed at room temperature (RT) and washing was performed twice with H₂O.

Subsequently, the number of colonies including more than 50 cells was measured, and the colony formation efficiency of the cells was calculated. The colony formation efficiency of the cells was calculated by dividing the number of colonies per dish by the number of cells (630) inoculated per dish. The results are shown in Table 1. Note that the values in Table 1 each show the average ±SD (n=3).

TABLE 1
Colony formation efficiency of cells (%)

| | |
|---|---|
| 0.1% gelatin coat | 21.1 ± 3.3 |
| without gelatin coat | 23.1 ± 3.7 |

As shown in Table 1, as a result of the CFU assay, it was found that the obtained npBM-MSC can form colonies regardless of the presence or absence of the gelatin coat.

### [Average Diameter of Cells]

With respect to hBM-MSC (P4) and the obtained npBM-MSC, the results of measurement of the average diameter of the cells are shown in Table 2. The average diameter of the cells was measured using Nucleo Counter NC-200 (trademark), and the average value (n=3) was calculated.

TABLE 2
Average diameter of cells

| | |
|---|---|
| npBM-MSC | 13.2 μm |
| hBM-MSC | 17.5 μm |

As shown in Table 2, it was found that the obtained neonatal pig bone marrow-derived mesenchymal stem cells have a smaller average diameter than the human bone marrow-derived mesenchymal stem cells.

### [Evaluation of Growth Rate]

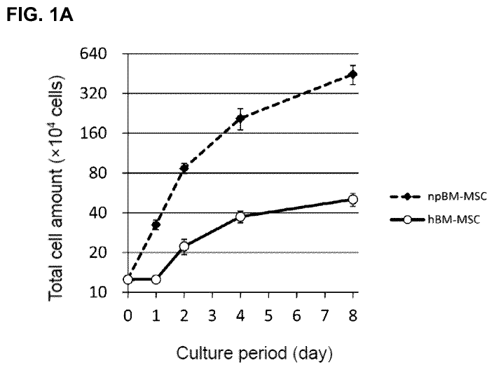

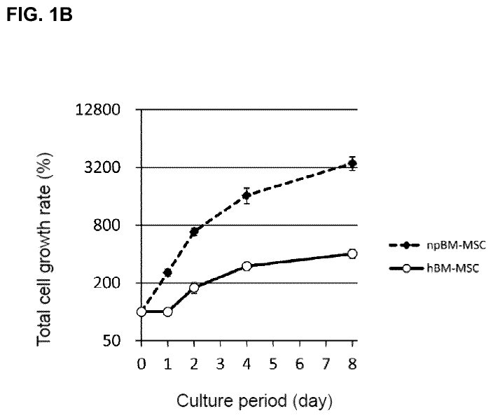

With respect to the hBM-MSC and the npBM-MSC, the cells were inoculated into a T25 flask at a density of 5000 cells/cm² (1.25×10⁵ cells/flask) and cultured using the MSC medium. The MSC medium was replaced with a fresh one every three days. After 1, 2, 4, and 8 days from the start of culture, the total numbers of viable cells and dead cells were counted. The results are shown in Table 3 and Table 4, and also FIG. 1A and FIG. 1B. Note that the values in Table 3 and Table 4 are each the average ±SD (n=4).

TABLE 3
Total cell amount (×10⁴ cells)

| | Immediately | | | | |
|---|---|---|---|---|---|
| Group | after suspension | Day 1 | Day 2 | Day 4 | Day 8 |
| npBM-MSC | 12.7 | 32.5 ± 2.7 | 86.9 ± 7.7 | 207.9 ± 38.2 | 448.0 ± 73.6 |
| hBM-MSC | 12.5 | 12.5 ± 0.8 | 22.3 ± 2.9 | 37.4 ± 3.8 | 50.5 ± 5.7 |

TABLE 4
Total cell growth rate (%)

| | Immediately | | | | |
|---|---|---|---|---|---|
| Group | after suspension | Day 1 | Day 2 | Day 4 | Day 8 |
| npBM-MSC | 100 | 257 ± 22 | 687 ± 61 | 1643 ± 302 | 3542 ± 581 |
| hBM-MSC | 100 | 100 ± 7 | 178 ± 23 | 299 ± 31 | 404 ± 46 |

As shown in Table 3 and Table 4, and also in FIG. 1A and FIG. 1B, it was found that the obtained neonatal pig bone marrow-derived mesenchymal stem cells have a significantly higher growth rate than the human bone marrow-derived mesenchymal stem cells.

[Differentiation into Adipocytes]

With respect to the hBM-MSC and the npBM-MSC, differentiation into adipocytes was induced using hMSC differentiation BulletKit (trademark)-adipogeni, PT-3004 (manufactured by Lonza Walkersville, Inc.) according to the protocol. Staining was performed using Oil Red manufactured by Sigma-Aldorich Co. LLC on day 17 after the start of induction. As a result, it was found that the obtained neonatal pig bone marrow-derived mesenchymal stem cells can differentiate into adipocytes in the same manner as human bone marrow-derived mesenchymal stem cells.

[Differentiation into Osteocytes]

With respect to the hBM-MSC and the npBM-MSC, differentiation into osteocytes was induced using hMSC differentiation BulletKit (trademark)-osteogenic, PT-3002 (manufactured by Lonza Walkersville, Inc.) according to the protocol. Staining was performed using an alkaline phosphatase staining kit manufactured by Cosmo Bio Co., Ltd. on day 14 after the start of induction to confirm differentiation into bone cells. As a result, it was found that the obtained neonatal pig bone marrow-derived mesenchymal stem cells can differentiate into osteocytes in the same manner as human bone marrow-derived mesenchymal stem cells.

[Differentiation into Chondrocytes]

With respect to the npBM-MSC, differentiation into osteocytes was induced using hMSC differentiation BulletKit (trademark)-chondrogenic, PT-3003 (manufactured by Lonza Walkersville, Inc.) according to the protocol. HE staining was performed on day 19 after the start of induction. As a result, it was found that the obtained BM-MSC could be differentiated into chondrocytes.

### Reference Example 2

[Culture of Cells in npMNC Fraction and Preparation of npBM-MSC]

The MSC minimal essential medium or the MSC medium was left to stand for 10 to 15 minutes in an incubator (37° C., 5% CO₂) before use. In the same manner as in Test Example 1, the cell suspension containing the cells in the npMNC fraction cryopreserved in a cryovial was promptly thawed in a water bath at 37° C. The thawed cell suspension was calmly added to 30 mL of the MSC minimal essential medium with temperature-equilibrated to 37° C., and the resultant was dispensed into two 50-mL tubes in an amount of 15 mL each.

Centrifugation was performed at room temperature for 5 minutes at 500×g, and the resulting pellet was resuspended in 2 mL of the temperature-equilibrated MSC minimal essential medium or MSC medium, and the suspension was gently pipetted up and down. The results of measurement of the total number of cells and the number of viable cells are shown below.
2 mL of the MSC minimal essential medium: the total number of cells: 2.60×10⁶, the number of viable cells: 4.8×10⁵, and the viability: 18.5%
2 mL of the MSC medium: the total number of cells: 2.55×10, the number of viable cells: 4.5×10⁵, and the viability: 17.6%

The cell suspension in an amount calculated so that the number of inoculated cells is as described below was added to a 6-well plate (without gelatin coat) in which the following medium was placed in each well, and the cell suspension was dispersed on a growth surface by gently shaking.
2 mL of the MSC minimal essential medium: the cells were inoculated at 2.60×10⁶ cells/well
2 mL of the MSC medium: the cells were inoculated at 2.55×10⁶ cells/well

The plate was placed in a CO₂ incubator, and incubated under the conditions of 37° C., 5% CO₂, and 90% humidity. After 3 days and after 6 days from the inoculation, the medium was replaced with the MSC medium to proliferate the cells, and on day 8 after the inoculation, the cells were subcultured.

### [Subculture]

After the npBM-MSC reached nearly 50% to 60% confluence, the cells were recovered from one well and reinoculated into a T75 flask without gelatin coat.

The cells were washed with 2 mL of PBS (−), 320 μL of 0.25% trypsin was added per well, and the cells were left to stand for a few minutes in an incubator, after the cells were peeled off, the medium was neutralized with 1680 μL of the MSC medium. The cell suspension was collected in a 50-mL tube, and 8 mL of the MSC medium was added thereto, followed by centrifugation at room temperature for 5 minutes at 500×g.

To the obtained pellet, the temperature-equilibrated MSC medium (2 mL) was added, and the pellet was gently resuspended therein by pipetting up and down, and the results of measurement of the total number of cells and the number of viable cells are shown below:
a group of the MSC minimal essential medium at the time of inoculation of P0: the total number of cells: 5.0×10⁵, the number of viable cells: 5.0×10⁵, and the viability: 100%
a group of the MSC medium at the time of inoculation of P0: the total number of cells: 3.3×10⁵, the number of viable cells: 3.3×10⁵, and the viability: 100%

To a T75 flask (without gelatin coat), the MSC medium (15 mL) was added, the neonatal pig bone marrow-derived mesenchymal stem cells (npBM-MSC) were reinoculated into the flask such that the number of cells became as described below; and cultured in an incubator. The cells were defined as a first subculture.
a group of the MSC minimal essential medium at the time of inoculation of P0: the number of viable cells: 5.0×10⁵ cells/flask
a group of the MSC medium at the time of inoculation of P0: the number of viable cells: 3.3×10⁵ cells/flask
   [Preparation of npBM-MSC]

After the reinoculated cells reached nearly 80% to 90% confluence according to the above-mentioned procedure, the cells were collected from one flask of the T75 flasks (without gelatin coat). The cells were washed with 8 mL of PBS (−), 2.4 mL of trypsin was added at 0.25 mL/well, and the cells were left to stand for a few minutes in an incubator, after the cells were peeled off, the medium was neutralized with 12.6 mL of the MSC medium. The cell suspension was collected in a 50-mL tube, followed by centrifugation at room temperature for 5 minutes at 500×g.

To the obtained pellet, the temperature-equilibrated MSC medium (5 mL) was added, and the pellet was calmly resuspended by pipetting up and down, and the results of measurement of the total number of cells and the number of viable cells are shown below:

The cells from one flask (the MSC minimal essential medium for 3 days after the inoculation of P0): the total number of cells: 5.12×10⁶, the number of viable cells: 5.09×10⁶, and the viability: 99.5%

The cells from one flask (the MSC medium from the inoculation of P0): the total number of cells: 4.76×10⁶, the number of viable cells: 4.73×10⁶, and the viability: 99.4%

[Cryopreservation of npBM-MSC]

Apart from the above-mentioned culture, the early subculture cells were frozen in the same manner as in Test Example 1, whereby a cell stock was prepared.

### Reference Example 3

A cell surface antigen on the npMNC prepared in Reference Example 1 and Reference Example 2 was analyzed. The preparation method for each sample used in the analysis is shown in Table 5. In Table 5, “Switch” indicates that the culture was performed by using the MSC minimal essential medium (vitamin C-free) during initial culture, and changing the medium to the MSC medium (containing vitamin C) that is a growth medium during proliferation culture.

TABLE 5
Preparation method for each sample

| | | 0.1% gelatin coat | | |
|---|---|---|---|---|
| | | during | during | |
| Sample | | initial | proliferation | |
| No. | Medium | culture | culture | Freezing condition |
| 1 | Switch | with | with | CELLBANKER |
| | | | | (registered trademark) |
| 2 | Switch | with | with | OPF-301 (10% DMSO) |
| 3 | Switch | with | without | CELLBANKER |
| | | | | (registered trademark) |
| 4 | Switch | with | without | OPF-301 (10% DMSO) |
| 5 | Switch | without | without | OPF-301 (10% DMSO) |
| 6 | Only MSC | without | without | OPF-301 (10% DMSO) |
| | medium | | | |

### [Analysis of Cell Surface Antigen]

Each cell sample was taken out from the liquid nitrogen tank, the cap was loosened to release the pressure, and the cap was closed again, and then, the sample was thawed in a thermostat bath preheated to 37° C. while lightly stirring for 1 minute to 2 minutes. Each thawed cell was transferred to a 15-mL centrifuge tube containing 5 mL of Stain Buffer (manufactured by BD), followed by centrifugation at 4° C. for 5 minutes at 500×g, and the supernatant was removed. Stain Buffer (5 mL) was added thereto, followed by centrifugation at 4° C. for 5 minutes at 500×g, and washing was performed twice.

The cells were resuspended in 2 mL of Stain Buffer (manufactured by BD), and the number of viable cells was counted. Centrifugation was performed again (500×g, 5 minutes, 4° C.), and the cells were resuspended in Stain Buffer (manufactured by BD) so that the cell density became 1×10⁷ cells/mL, and the cell suspension was dispensed into 1.5-mL tubes in an amount of 20 μL (cell count: 2×10⁵ cells) each, whereby four tubes in total were prepared for each of the following samples: unstained control, CD44, CD90, and Isotype Control.

To the corresponding tubes, 4 μL of Anti-CD44, Mouse (MEM-263), PE (manufactured by GeneTex, Inc.), 1 μL of PE Mouse Anti-Human CD90 (manufactured by BD) (having cross-reactivity with pig), and 4 μL of PE Mouse IgG1, κ Isotype Control (manufactured by BD) were added, followed by incubation for 45 minutes on ice under shading. The unstained control was also stored on ice.

To each tube, 1 mL of Stain Buffer (manufactured by BD) was added, followed by centrifugation at 4° C. for 5 minutes at 500×g, and washing was performed twice. The cell pellet was loosened by tapping and resuspended in 500 μL of Stain Buffer (manufactured by BD), and the suspension was passed through a filter immediately before the analysis and transferred to a test tube for flow cytometry: The sample was stored on ice under shading until the analysis, and analyzed using flow cytometry.

As a result, any sample was positive for CD44 and CD90 that are markers for mesenchymal stem cells. In addition, it is considered that objective mesenchymal stem cells could be established even without performing coating with gelatin during initial culture. Note that in any case, a nonspecific reaction was not observed in the measurement for the Isotype Control.

### Reference Example 4

### [Preparation of Neonatal Porcine Pancreatic Islet-Derived Mesenchymal Stem Cells]

The pancreatic islet was collected from a neonatal pig, and was subjected to suspension culture, thereby preparing a cell cluster, the cell cluster was cryopreserved in the same manner as in Reference Example 1. The neonatal porcine pancreatic islet cryopreserved in a cryovial was promptly thawed in a water bath at 37° C.

The thawed pancreatic islet suspension was calmly added to 30 mL of the MSC minimal essential medium adjusted to reach the temperature equilibrium (37° C.) using a micropipette. The resultant was centrifuged at 4° C. for 1 minute at 210×g. Note that in the case where the pancreatic islet was not frozen, after the pancreatic islet was precipitated at room temperature by free falling, the supernatant was removed. The resulting pellet was resuspended in 4 mL of the temperature-equilibrated MSC minimal essential medium, and the suspension was gently pipetted up and down.

To a 6-well plate, the pancreatic islet suspension was added, and the cell suspension was dispersed on a growth surface (without gelatin coat) by gently shaking, inoculation was performed in 2 mL of the MSC minimal essential medium at the pancreatic islet/well in the range of 1650 IEQ to 2125 IEQ.

In a CO₂ incubator, culture was performed under the conditions of 37° C., 5% CO₂, and 90% humidity, and after 3 days, the medium was replaced with the MSC medium so as to proliferate the cells, and thereafter, the MSC medium was replaced with a fresh one every three days. The preparation conditions for the samples are shown in Table 6. After 6 days from the inoculation, 100% confluence was reached regardless of whether initial freezing was performed or not performed.

TABLE 6

| | | | | Subculture | Liquid | Cell density |
|---|---|---|---|---|---|---|
| Sample | | | Freezing | number at | amount | (×10⁶ total |
| No. | Origin | Conditions | at P0* | freezing* | (mL) | cells/mL) |
| 7 | pancreatic | On the day of | without | P2 (OPF-301) | 1 | 2.23 |
| | islet | preparing pancreatic, | | | | |
| | | islet (day 1) | | | | |
| 8 | pancreatic | On day 3 after | without | P2 (OPF-301) | 1 | 1.22 |
| | islet | preparing pancreatic | | | | |
| | | islet (day 4) | | | | |
| 9 | pancreatic | On the day of | with | P2 (OPF-301) | 1 | 3.25 |
| | islet | preparing pancreatic | (OPF-301) | | | |
| | | islet (day 1) | | | | |
| 10 | pancreatic | On day 3 after | with | P2 (OPF-301) | 1 | 2.3 |
| | islet | preparing pancreatic | (OPF-301) | | | |
| | | islet (day 4) | | | | |

*The type of 10% DMSO is shown in parentheses.

### [Subculture]

After the neonatal porcine pancreatic islet-derived mesenchymal stem cells (npISLET-MSC) reached about 80% to nearly 95%, the cells were recovered from two wells and reinoculated into a T75 flask without gelatin coat.

The cells were washed with 2 mL of PBS (not containing calcium and magnesium), 320 μL of 0.25% trypsin was added per well, and the cells were left to stand for a few minutes in an incubator, after the cells were peeled off, the medium was neutralized with 1680 μL of the MSC medium. The cell suspension was collected in a 50-mL tube using a 1-mL pipette, and 16 mL (8 mL×2 wells) of the MSC medium was added thereto, followed by centrifugation at room temperature for 5 minutes at 500×g. The obtained pellet was gently resuspended in the temperature-equilibrated MSC medium (2 mL) using a pipette

### [Average Diameter of Cells]

The MSC medium (20 mL) was added to T75 flasks without gelatin coat, and reinoculation was performed, the cells were cultured under the conditions of 37° C., 5% CO₂, and 90% humidity in a CO₂ incubator. The cells were defined as a first subculture. After 3 days from the inoculation of the first subculture, 100% confluence was reached regardless of whether initial freezing was performed or not performed. From this, it was found that the growth rate of mesenchymal stem cells prepared from the pancreatic islet of a neonatal pig is equivalent to that of mesenchymal stem cells prepared from the bone marrow of a neonatal pig. The average diameters of the obtained neonatal porcine pancreatic islet-derived mesenchymal stem cells are shown in Table 7.

TABLE 7

| | Sample No. | | | |
|---|---|---|---|---|
| | 11 | 12 | 13 | 14 |
| Preparation | without freezing | with freezing | without freezing on | with freezing on |
| conditions | immediately | immediately | day 3 of culture | day 3 of culture |
| | after preparing | after preparing | after preparing | after preparing |
| | pancreatic islet | pancreatic islet | pancreatic islet | pancreatic islet |
| Type of 10% | — | OPF-301 | — | OPF-301 |
| DMSO solution | | | | |
| at freezing | | | | |
| Average diameter | 12.1 μm | 12.2 μm | 12.0 μm | 12.3 μm |

As shown in Table 7, it was found that neonatal porcine pancreatic islet-derived mesenchymal stem cells can be prepared regardless of the freezing condition in the preparation of the pancreatic islet, and the average diameters are equivalent regardless of whether freezing is performed or not performed.

### [Analysis of Cell Surface Antigen]

Each cell sample was taken out from the liquid nitrogen tank, the cap was loosened to release the pressure, and the cap was closed again, and then, the sample was thawed in a thermostat bath preheated to 37° C. while lightly stirring for 1 minute to 2 minutes. Each thawed cell was transferred to a 15-mL centrifuge tube containing 5 mL of Stain Buffer (manufactured by BD), followed by centrifugation at 4° C. for 5 minutes at 500×g, and the supernatant was removed. Stain Buffer (5 mL) was added thereto, followed by centrifugation at 4° C. for 5 minutes at 500×g, and washing was performed twice.

The cells were resuspended in 2 mL of Stain Buffer (manufactured by BD), and the number of viable cells was counted. Centrifugation was performed again (500×g, 5 minutes, 4° C.), and the cells were resuspended in Stain Buffer (manufactured by BD) so that the cell density became 1×10⁷ cells/mL, and the cell suspension was dispensed into 1.5-mL tubes in an amount of 20 μL (cell count: 2×10⁵ cells) each, whereby four tubes in total were prepared for each of the following samples: unstained control, CD29, CD44, and CD90.

To the corresponding tubes, 1 μL of Mouse Alexa Fluor 647 Mouse Anti-Pig CD29 (manufactured by BD), 4 μL of Anti-CD44, Mouse (MEM-263), PE (manufactured by GeneTex, Inc.), and 1 μL of PE Mouse Anti-Human CD90 (manufactured by BD) (having cross-reactivity with pig) were added, followed by incubation for 45 minutes on ice under shading. The unstained control was also stored on ice.

To each tube, 1 mL of Stain Buffer (manufactured by BD) was added, followed by centrifugation at 4° C. for 5 minutes at 500×g, and washing was performed twice. The cell pellet was loosened by tapping and resuspended in 500 μL of Stain Buffer (manufactured by BD), and the suspension was passed through a filter immediately before the analysis and transferred to a test tube for flow cytometry: The sample was stored on ice under shading until the analysis, and analyzed using flow cytometry.

As a result, in any sample, a high positive rate was observed for CD29, CD44, and CD90 that are markers for mesenchymal stem cells. In addition, it is considered that objective mesenchymal stem cells could be established regardless of whether freezing is performed or not performed at initial culture.

### Test Example 1

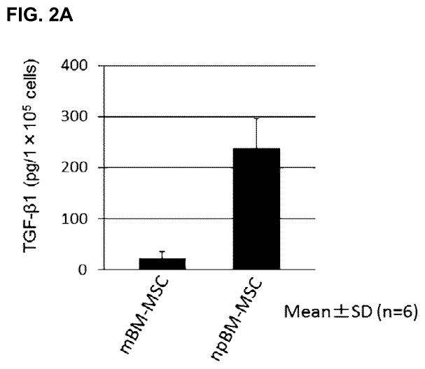

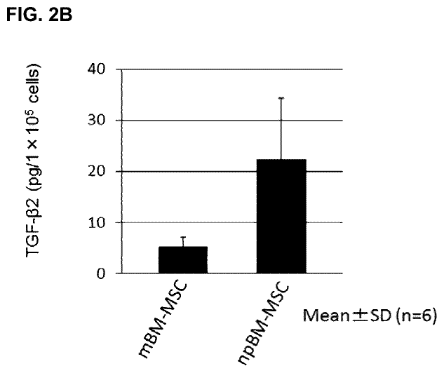

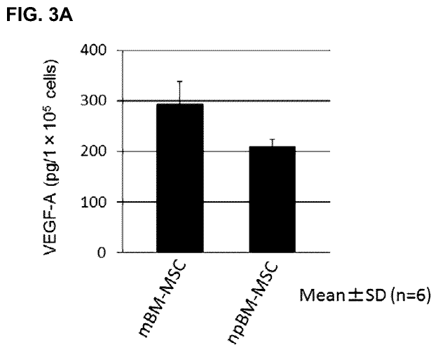

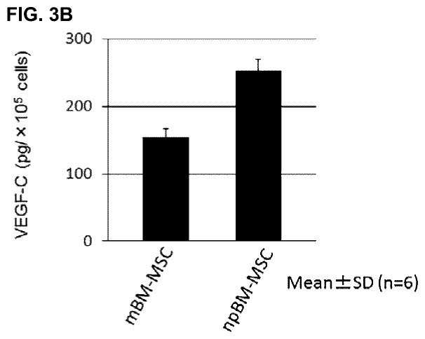

In a 6-well plate, npBM-MSC prepared in the same manner as in Reference Example 1 was inoculated at a cell count of 5×10⁴ cells/2 mL/well or mBM-MSC (OriCell™ strain C57BL/6 mice, catalog number MUB MX-01001, lot number 170221 I31, Cyagen Biosciences, Inc.) was inoculated at a cell count of 1×10⁵ cells/2 mL/well, and cultured using an MSC medium. After the culture for 3 days, the supernatant was collected, and the concentrations of TGF-β1, TGF-β2, VEGF-A, and VEGF-C were measured. The concentrations of TGF-β1 and TGF-β2 were measured using an ELISA kit and corrected by the number of cells at the time of supernatant recovery. The results are shown in FIGS. 2(a) and 2(b) and FIGS. 3(a) and 3(b). The concentration of TGF-β1 of each of the pigs and mice was measured using R&D SYSTEMS (registered trademark) Quantikine (registered trademark) ELISA Mouse/Rat/Porcine/Canine TGF-β1 (MB100B, Bio-Techne Corporation, Minneapolis, MN, USA). The concentration of TGF-β2 of each of the pigs and mice was measured using R&D SYSTEMS (registered trademark) Quantikine (registered trademark) ELISA Mouse/Rat/Canine/Porcine TGF-β2 (MB200, Bio-Techne Corporation). The concentrations of VEGF-A of the pigs and VEGF-A of the mice were respectively measured using a Swine VEGF-A Do-It-Yourself ELISA (KFS-DIY0751S-003, Kingfisher Biotech, Inc., St. Paul, MN, USA) and a Mouse VEGF-A Do-It-Yourself ELISA (KFS-DIY0746M-003, Kingfisher Biotech, Inc.). The concentrations of VEGF-C the pigs and the VEGF-C of mice were measured by using a Porcine VEGF-C ELISA kit (MBS2512025, My BioSource, Inc. San Diego, CA, USA), and a Mouse VEGF-C ELISA kit (MBS2503462, MyBioSource, Inc.).

As shown in FIGS. 2(a) and 2(b), FIGS. 3(a) and 3(b), it was found that the neonatal pig bone marrow-derived mesenchymal stem cells produce TGF-β1, TGF-β2, VEGF-A, and VEGF-C. In addition, it has been found that the neonatal pig bone marrow-derived mesenchymal stem cells exhibit a high expression of TGF-β1, TGF-β2, and VEGF-C, as compared with mouse bone marrow-derived mesenchymal stem cells.

### Test Example 2

The left femoral artery of a 12-week-old male C57BL/6J mouse was ligated and then dissected to prepare an ischemic limb according to the method described in the literature (Motohiro Nishida, et al: J Vasc Surg: 2016: 64:219-226). In the thigh muscle tissue of the prepared ischemic limb, the neonatal pig bone marrow-derived mesenchymal stem cells prepared in the same manner as in Reference Example 1 were suspended in PBS so as to have a cell number of 1×10⁵ cells/0.1 mL, 5×10⁵ cells/0.1 mL, 1×10⁶ cells/0.1 mL, or 2.5×10⁶ cells/0.1 mL, and 0.1 mL was intramuscularly injected.

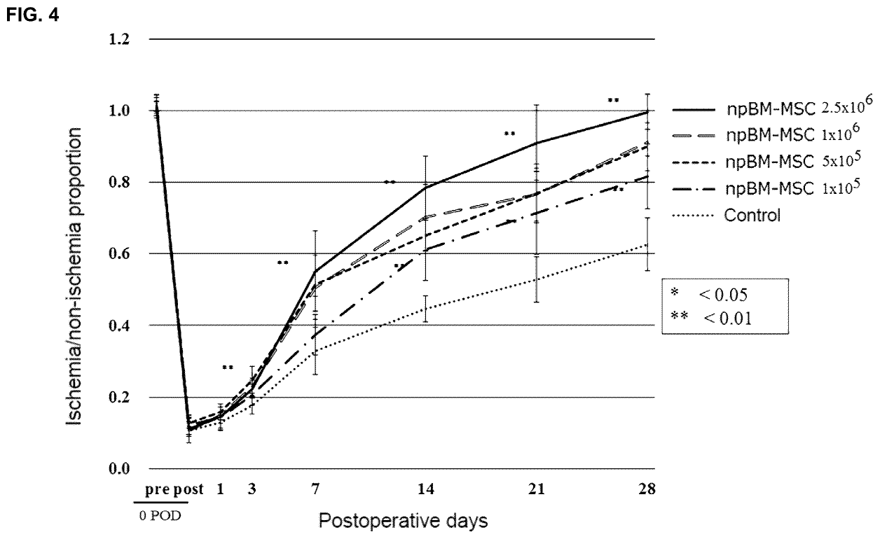

Using a laser Doppler velocimeter (manufactured by Moor Instruments Ltd., DS2 manufactured by UK), the blood flow of the lower limb was measured over time up to 4 weeks after surgery, and the affected side and the healthy (control) side were compared. The results are shown in FIG. 4.

As shown in FIG. 4, a significant blood flow improvement effect was obtained by the administration of neonatal pig-derived mesenchymal stem cells.

### Test Example 3

In the thigh muscle tissue of the ischemic limb prepared in the same manner as in Test Example 2, the neonatal pig bone marrow-derived mesenchymal stem cells or mouse bone marrow-derived mesenchymal stem cells prepared in the same manner as in Reference Example 1 were suspended in PBS so as to have a cell number of 1×10⁵ cells/0.1 mL, or 1×10⁶ cells/0.1 mL, and 0.1 mL was intramuscularly injected.

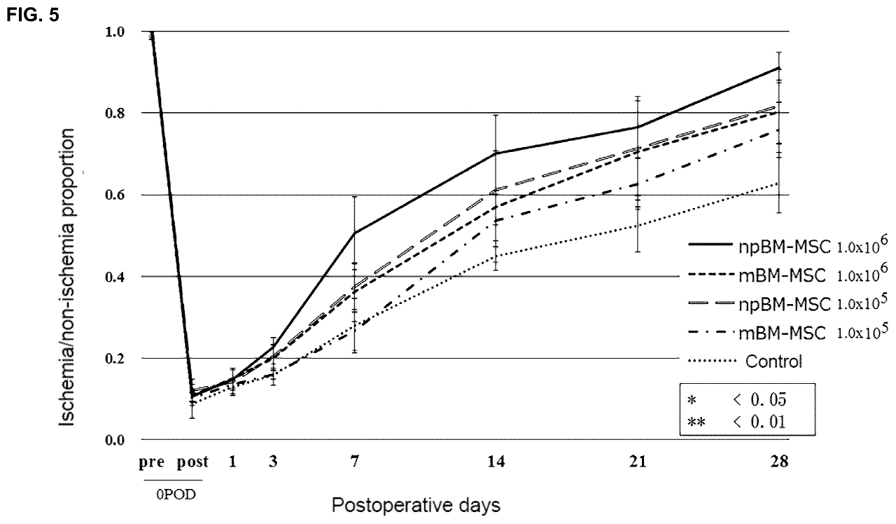

Using a laser Doppler velocimeter (manufactured by Moor Instruments Ltd., DS2 manufactured by UK), the blood flow of the lower limb was measured over time up to 4 weeks after surgery, and the affected side and the healthy (control) side were compared. The results are shown in FIG. 5.

As shown in FIG. 5, it was found that a blood flow improvement effect by neonatal pig-derived mesenchymal stem cells was significantly higher than that of mouse bone marrow-derived mesenchymal stem cells.

Although the invention has been described in detail with reference to specific embodiments, it will be apparent to those skilled in the art that various changes and modifications can be made without departing from the spirit and scope of the invention. This application is based on a Japanese patent application (Japanese Patent Application No. 2019-82768) filed on Apr. 24, 2019, the contents of which are incorporated herein by reference in its entirety.

1. A method for treating a non-porcine animal, comprising:
administering an effective amount of a neonatal pig-derived mesenchymal stem cell which produces at least one humoral factor selected from a transformation growth factor-β (TGF-β) 1, TGF-β2, a vascular endothelial growth factor (VEGF)-A and VEGF-C to the non-porcine animal.

2. The method according to claim 1, wherein the non-porcine animal is treated by the promotion of angiogenesis and/or lymphangiogenesis.

3. The method according to claim 1, wherein at least one selected from peripheral artery diseases, cerebral infarction, myocardial infarction, acute lung injury, wounds, and skin injury is treated.

4. The method according to claim 1, wherein the neonatal pig-derived mesenchymal stem cell is derived from a fetal pig or a pig less than one month after birth.

5. The method according to claim 1, wherein the neonatal pig-derived mesenchymal stem cell is derived from a fetal pig or a pig less than 25 days after birth.

6. The method according to claim 1, wherein the non-porcine animal is a human.

7. The method according to claim 1, wherein the neonatal pig-derived mesenchymal stem cell is obtained by collecting cells from a neonatal pig, freezing the collected cells, thawing the frozen cells, inoculating and culturing the thawed cells to obtain primary cultured cells, subculturing the primary cultured cells 3 to 12 days after the inoculation.

8. The method according to claim 1, wherein the neonatal pig-derived mesenchymal stem cell has an average diameter of 17 μm or less.

9. The method according to claim 1, wherein doubling time in a logarithmic growth phase of the neonatal pig-derived mesenchymal stem cell is 36 hours or less.
